(19) United States
(12) Patent Application Publication
(10) Pub. No.: US 2021/0346332 A1
(43) Pub. Date: Nov. 11, 2021

(54) COMPOSITIONS AND METHODS FOR TREATING AND PREVENTING LEBER'S HEREDITARY OPTIC NEUROPATHY

(71) Applicant: IMBRIA PHARMACEUTICALS, INC., Boston, MA (US)

(72) Inventors: Andrew D. Levin, Newton, MA (US); David-Alexandre Gros, Rancho Santa Fe, CA (US)

(21) Appl. No.: 17/282,587

(22) PCT Filed: Oct. 10, 2019
(86) PCT No.: PCT/US2019/055666

§ 371 Date: Apr. 2, 2021

Related U.S. Application Data
(60) Provisional application No. 62/744,242, filed on Oct. 11, 2018.

(51) Int. Cl. A61K 31/225; A61K 31/194; A61K 47/18; A61K 47/10

(52) U.S. Cl. CPC A61K 31/225; A61K 47/10; A61K 47/183; A61K 31/194

(57) ABSTRACT

Compositions and methods for treating and preventing Leber's hereditary optic neuropathy are provided.

### CROSS-REFERENCE TO RELATED APPLICATION

This application claims the benefit of, and priority to, U.S. Provisional Application No. 62/744,242, filed Oct. 11, 2018, the contents of which are incorporated herein by reference.

### FIELD OF THE INVENTION

This application is related to compositions and methods for treating and preventing Leber's hereditary optic neuropathy.

### BACKGROUND

Leber's hereditary optic neuropathy (LHON) is an inherited condition in which retinal degeneration leads to loss of vision. Onset of vision loss due to LHON typically occurs in early adulthood and leads to visual acuity of 20/200 or worse within months. Although the prevalence of LHON has not been studied widely, the incidence of LHON in certain European populations is about 1 in 50,000 people, suggesting that hundreds of thousands of people may be affected worldwide. LHON is caused by any of several mitochondrial mutations that impair function of NADH dehydrogenase, an enzyme involved in mitochondrial production of the high-energy molecule adenosine triphosphate (ATP). More than 80% of LHON patients are male, but only women can transmit the condition to their children.

Existing treatments for LHON are extremely limited. When taken after the onset of vision loss due to LHON, the drug idebenone prevents further loss of vision in some patients. However, idebenone does not result in demonstrable improvement of vision in those patients and has no beneficial effect at all for others. Several other drugs, such as brimonidine, minocycline, curcumin, glutathione, and elamipretide, and other treatment strategies, such as near infrared light treatment and viral vector techniques, are still under investigation for treatment of LHON. Other approaches for managing LHON merely involve behavioral changes designed to minimize vision loss. For example, LHON patients are advised to avoid tobacco, alcohol, and certain prescription medicines that are generally toxic to the optic nerve. Thus, despite our understanding of the molecular basis of LHON, we lack tools to combat the disease, and many people continue to suffer permanent vision loss as a result.

### SUMMARY

The invention provides methods for treating and preventing LHON by providing compositions that promote mitochondrial production of ATP in the absence of NADH dehydrogenase activity. The compositions include water-soluble compounds that are metabolized in the body to release TCA intermediates, such as succinate. Because succinate does not depend on NADH dehydrogenase to contribute electrons to the mitochondrial electron transport chain, it drives mitochondrial ATP synthesis in individuals with LHON-causing mutations. Therefore, the methods of the invention compensate for the metabolic deficiency in such individuals to prevent or alleviate retinal degeneration and vision loss.

The methods of the invention employ conjugates of TCA cycle intermediates that have higher solubility compared to unadulterated intermediates. In preferred embodiments, the methods use TCA cycle intermediates that are conjugated to amino acids, such as serine and tyrosine. Consequently, the compounds are non-toxic, readily absorbed, and circulate freely throughout the body. Thus, compared to methods involving administration of free TCA cycle intermediates, the methods of the invention allow delivery of succinate at higher quantities to achieve greater therapeutic benefit.

The methods allow delivery of the compounds by various routes of administration. For example, the compounds may be delivered intraocularly to directly target the tissue affected in LHON. The high solubility of the compounds also makes them suitable for intravenous injection and other methods of systemic administration. In addition, the compounds can be provided orally because the covalent linkage eliminates the offensive tastes and odors produced by free TCA cycle intermediates.

In an aspect, the invention provides methods of treating or preventing Leber's hereditary optic neuropathy in a subject by providing to a subject having or at risk of developing Leber's hereditary optic neuropathy a composition containing a compound that contains one or more TCA cycle intermediates or prodrugs thereof and one or more capping moieties. The compound may contain two or more TCA cycle intermediates or prodrugs thereof and/or two or more capping moieties.

The capping moiety may be an amino acid, a polyol, or another TCA cycle intermediate or prodrug thereof. The compound may have two or more capping moieties. For example, the compound may have two, three, four, five, or six capping moieties. The two or more capping moieties may be the same, or they may be different.

The capping moieties may be linked by any atoms on the TCA cycle intermediate or prodrug thereof. Preferably, capping moieties are substituted onto hydroxyl groups and attached via alkoxy linkages. Preferably, a capping moiety is substituted onto the hydroxyl group of each of the terminal carbon atoms in the carbon skeleton of the TCA cycle intermediate or prodrug thereof.

The polyol may be a C₂-C₂₀ polyol. The polyol may be glycerol. The polyol may be linked via a terminal hydroxy group or an internal hydroxy group. For example, glycerol may be linked to the TCA cycle intermediate or prodrug thereof via a hydroxy group on its first, second, or third carbon.

The amino acid may be a naturally-occurring amino acid. For example, the amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. Preferably, the amino acid is serine or tyrosine. Preferably, the serine or tyrosine is linked to the TCA cycle intermediate or prodrug thereof via the hydroxy group on its side chain. The amino acid may be a non-naturally-occurring amino acid.

Numerous TCA cycle intermediates or prodrugs thereof are known in the art, such as those described in PCT/US2017/019000, the content of which is incorporated by reference herein in its entirety. Any such compounds may be conjugated with one or more amino acids to improve the solubility, and therefore oral availability of those compounds. For example, the TCA cycle intermediate or prodrug thereof may be citrate, cis-aconitate, D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate or citrate. The TCA cycle intermediate may have L or R chirality. Compositions including such compounds may include only L-forms, only R-forms, or racemic mixtures of L- and R-forms of the TCA cycle intermediate.

The compound may include one or more atoms that are enriched for an isotope. For example, the compound may have one or more hydrogen atoms replaced with deuterium or tritium. The isotopically enriched atom or atoms may be located at any position within the compound.

The compound may have an octanol:water partition coefficient of less than 0.1, less than 0.01, less than 0.001, less than 0.0001, less than 0.0001, less than 0.00001, or less than 0.000001.

The compound may be or include succinate diserine, glycerol trisuccinate triserine, or glycerol trisuccinate trityrosine.

The compound may be or include a structure represented by one of formulas (I), (II), (III), (IV), (V), (VI), (VII), (VIII), (IX), (X), (XI), (XII), (XIII), (XIV), (XV), (XVI), (XVII), and (XVIII):

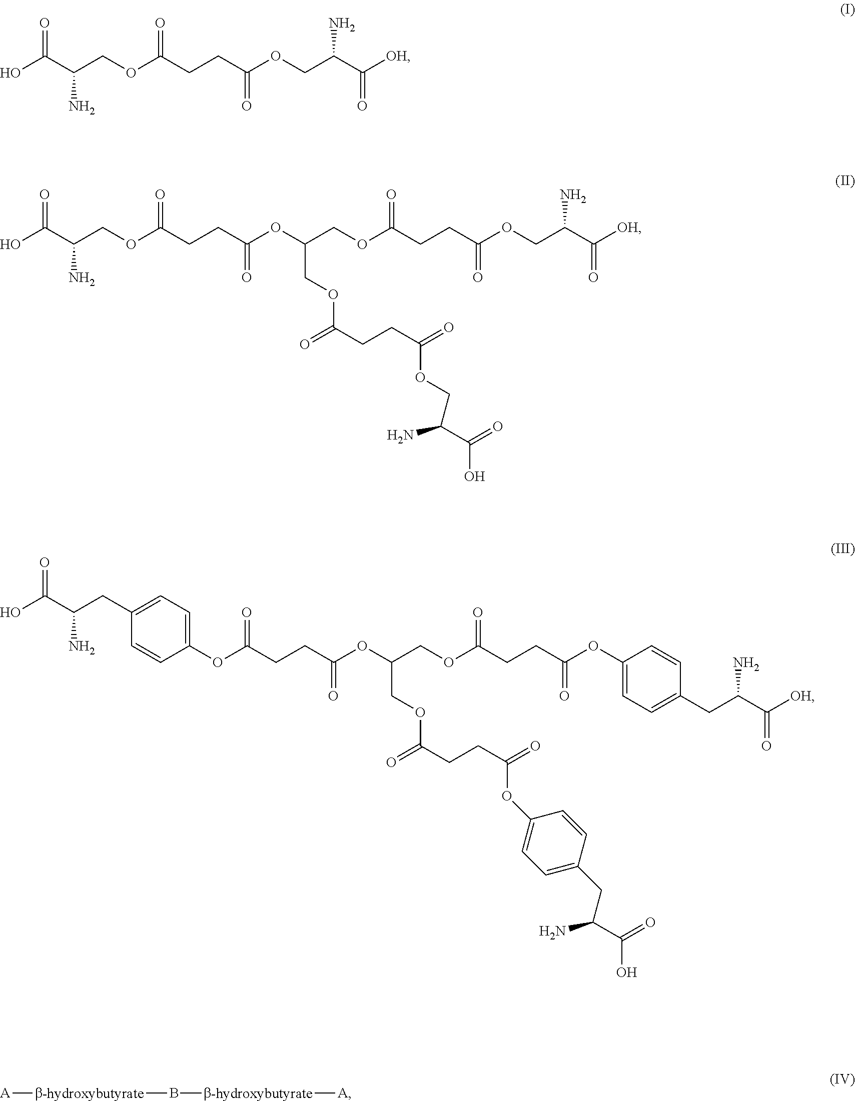

in which A is an amino acid, such as serine, and B is a TCA cycle intermediate, such as succinate,

C-D-E (V),

in which C is a first TCA cycle intermediate, such as malate, D is a second TCA cycle intermediate, such as succinate, and E is an amino acid, such as serine,

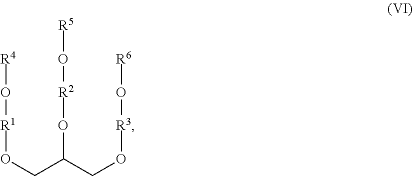

in which R¹, R², and R³ are TCA cycle intermediates or prodrugs thereof, and R⁴, R⁵, and R⁶ are amino acids,

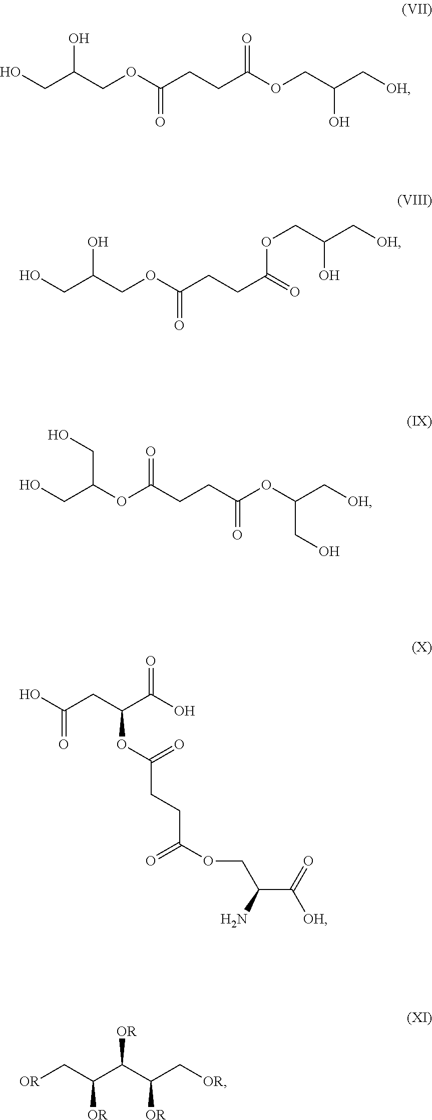

in which R is

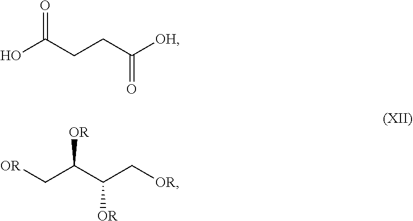

in which R is

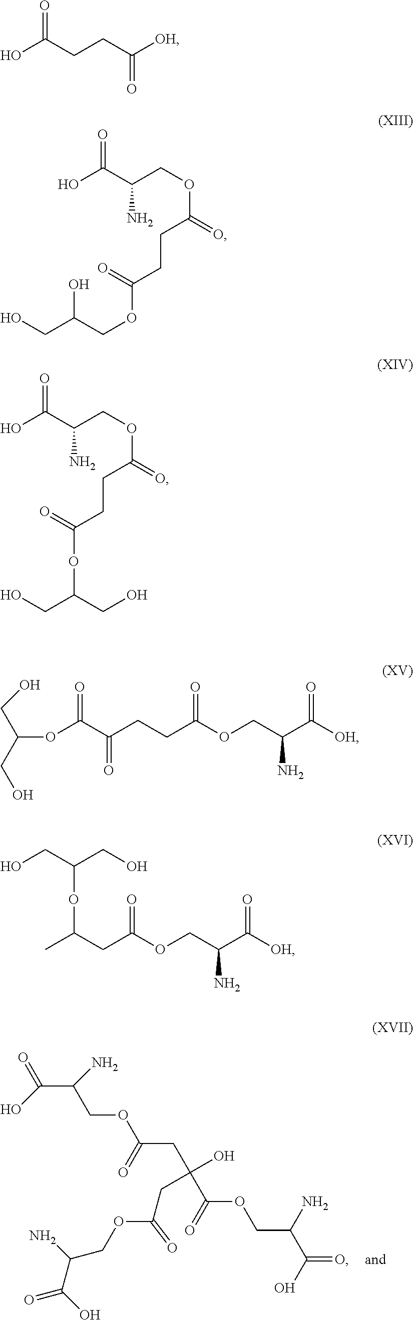

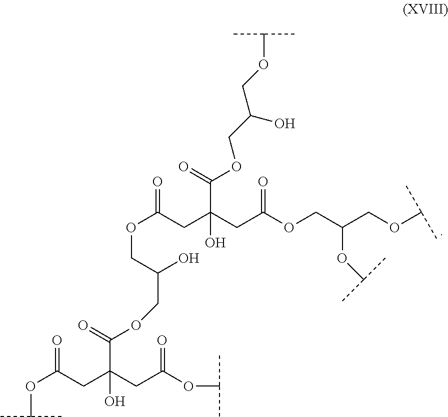

The methods may include providing the compound by any suitable route of administration. For example, the compound may be provided intraocularly, intravenously or orally.

The methods may include any suitable dosing regimen. For example, the compound may be provided in a single dose or in multiple doses. Multiple doses may be provided in provided separated by intervals, such as 4 hours, 6 hours, 8 hours, 12 hours, 24 hours, 2 days, 3 days, 4 days, 5 days, 1 week, 2 weeks, 3 weeks, 4 weeks, or more.

### DETAILED DESCRIPTION

The invention provides methods of treating and preventing Leber's hereditary optical neuropathy (LHON) by providing compositions that contain intermediates of the TCA cycle, such as succinate, that have been modified to improve their solubility. The compositions include compounds that comprise a TCA cycle intermediate conjugated to one or more amino acids, polyols, or both. The compounds can be cleaved in the body to release the intermediates to enter the TCA cycle to produce succinate, which can enter the mitochondrial electron transport chain. As a result, mitochondrial respiration is less dependent on NADH dehydrogenase, an enzyme that has reduced function in patients with mutations that cause LHON.

The TCA cycle is illustrated below:

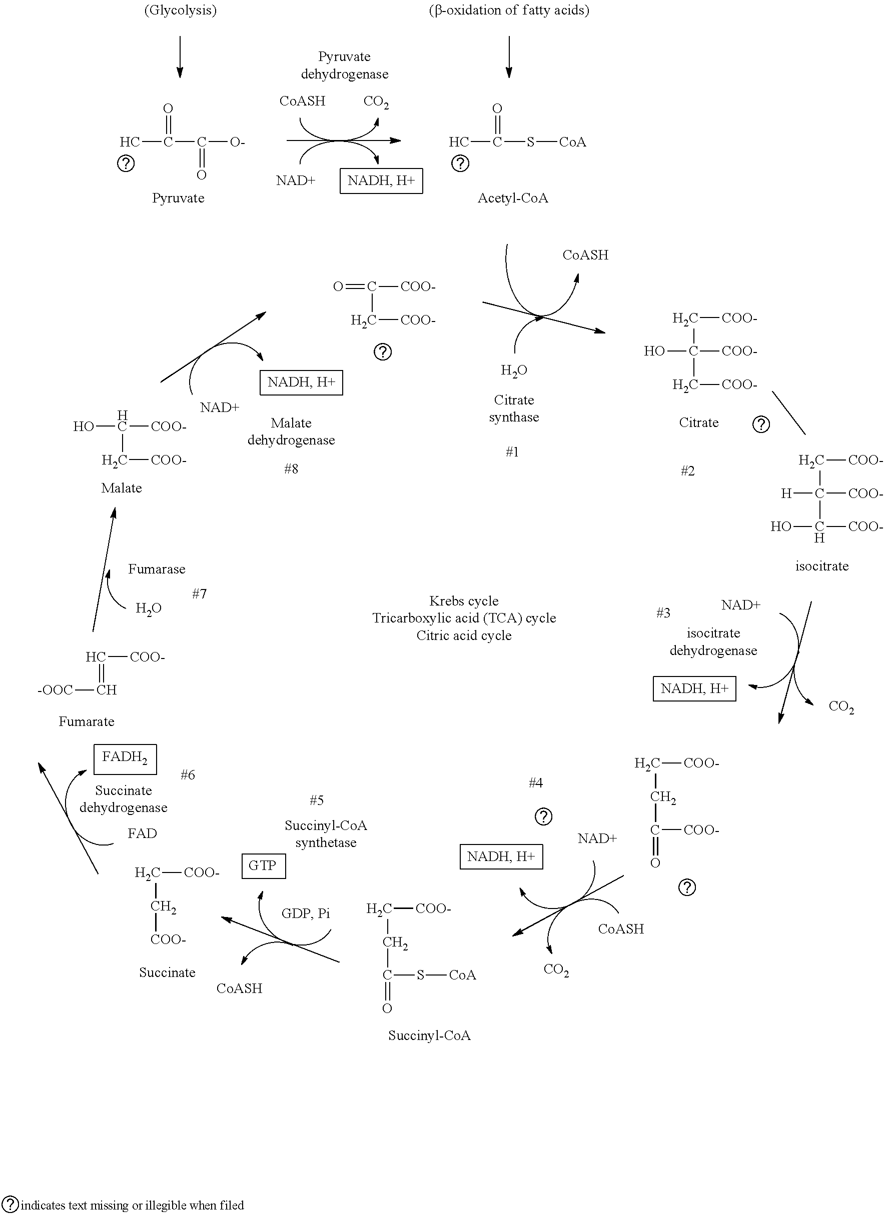

Another clinically important metabolic pathway is the mitochondrial electron transport chain. The electron transport chain uses a complex series of redox reactions to create a proton gradient across the mitochondrial inner membrane, and the chemiosmotic potential from the proton gradient is used to drive adenosine triphosphate (ATP) synthesis. The electron transport chain involves four enzymatic complexes in the mitochondrial inner membrane: NADH dehydrogenase, also called respiratory complex I; succinate dehydrogenase, also called respiratory complex II; coenzyme Q:cytochrome c reductase, also called respiratory complex III; and cytochrome c oxidase, also called respiratory complex IV. Electrons enter the transport chain in either of two ways. First, NADH dehydrogenase may transfer electrons from NADH to ubiquinone, the first intermediate electron carrier in the chain. Alternatively, electrons from succinate may be transferred to ubiquinone by succinate dehydrogenase. In the next step of the electron transport chain, electrons are transferred from ubiquinone to cytochrome c, the second intermediate electron carrier, by coenzyme Q:cytochrome c reductase. In the final step, cytochrome c oxidase transfers electrons from cytochrome c to molecular oxygen to form water, the net product of electron transport. Succinate dehydrogenase is the only enzyme that participates in both the TCA cycle and the electron transport chain.

Leber's hereditary optic neuropathy (LHON) is a retinal degenerative condition caused by defects in the electron transport chain. LHON results from mutations in mitochondrial genes that encode components of NADH dehydrogenase, such as MT-ND1, MT-ND4, MT-ND4L, and MT-ND6. Because mutations that cause LHON are encoded by genes in the mitochondrial genome, which is transmitted to the embryo from the egg but not from the sperm, LHON can only be inherited maternally.

An insight of the invention is that succinate is useful for treatment of LHON. Due to reduced NADH dehydrogenase activity, electron transport and ATP synthesis are decreased in patients with LHON. In particular, formation of ubiquinone, the first intermediate electron carrier in the chain, is diminished. However, electron transport activity can be restored by providing supplemental succinate, which can donate electrons to form ubiquinone via succinate dehydrogenase. Thus, providing additional succinate to serve as electron donor for the electron transport chain in patients with LHON compensates for the insufficiency of electron transfer from NADH.

Oral delivery of succinate or other TCA cycle intermediates in large quantities can be challenging because due to their strong taste and odor. Efforts have been made to identify modified forms of TCA cycle intermediates that are less malodorous for use in oral formulations. For example, compounds that contain a glycerol backbone linked to both succinate and fatty acids are disclosed in PCT/US2017/019000, which is incorporated herein by reference. However, such compounds are lipophilic and poorly soluble in water, which limits their bioavailability.

The methods provided herein use compounds that overcome the limited bioavailability of previously described compositions for delivery of TCA cycle intermediates. Such compounds are described in co-pending, co-owned International Application No. PCT/US2018/043487, the contents of which are incorporated herein in their entirety. Because the compounds are highly water soluble, they are absorbed and circulate readily in the body. In addition, the compounds can be cleaved to efficiently deliver TCA cycle intermediates to target tissues. Due to their superior bioavailability, the compounds of the invention can be provided in doses suitable for oral administration to treat abnormal TCA metabolism associated with a wide range of conditions.

The compounds include (1) one or more TCA cycle intermediates, metabolites that feed into the TCA cycle, such as pyruvate or ketone bodies, or prodrugs of TCA cycle intermediates or metabolites that feed into the TCA cycle and (2) one or more amino acids. Any of the TCA cycle intermediates described in the TCA cycle above may be used in compositions of the invention. In certain embodiments, any of the compounds described in PCT/US2017/019000 may be TCA cycle intermediates within the context of the invention.

A prodrug is a medication or compound that, after administration, is metabolized (i.e., converted within the body) into a pharmacologically active drug. The prodrug itself may be pharmacologically inactive. Prodrugs may be used to improve how a medicine is absorbed, distributed, metabolized, and excreted. The prodrug may improve the bioavailability of the active drug when the active drug is poorly absorbed from the gastrointestinal tract. The prodrug may improve how selectively the drug interacts with cells or processes that are not its intended target, thereby reducing unintended and undesirable side effects. The prodrug may be converted into a biologically active form (bioactivated) inside cells (a Type I prodrug) or outside cells (a Type II prodrug). The prodrug may bioactivated in the gastrointestinal tract, in systemic circulation, in metabolic tissue other than the target tissue, or in the target tissue.

Thus, the methods of the invention provide compounds that can be metabolized in the body to yield an intermediate of the TCA cycle, such as citrate, cis-aconitate, D-isocitrate, α-ketoglutarate, succinate, fumarate, malate, or oxaloacetate, or a molecule that can be metabolized to enter the TCA cycle, such as pyruvate or a ketone body. Examples of ketone bodies include acetone, acetoacetate, β-hydroxybutyrate, β-ketopentanoate, or β-hydroxypentanoate.

Any prodrugs of the TCA cycle intermediates described in the TCA cycle above may be used in methods of the invention. Any of the prodrugs or prodrugs of the compounds described in PCT/US2017/019000 may be TCA cycle intermediate prodrugs within the context of the invention.

The TCA cycle intermediate or prodrug thereof may include one or more substituents. The one or more substituents may be linked via, via any suitable chemical linkage, such as an alkoxyl linkage, to one or more carboxyl groups on the intermediate or prodrug thereof. The substituent may be a short-chain fatty acid, such as formate, acetate, propionate, butyrate, isobutyrate, valerate, or isovalerate.

The TCA cycle intermediate or prodrug may include succinate diserine, glycerol trisuccinate triserine, or glycerol trisuccinate trityrosine. The TCA cycle intermediate or prodrug may include a structure represented by one of formulas (I), (II) and (III):

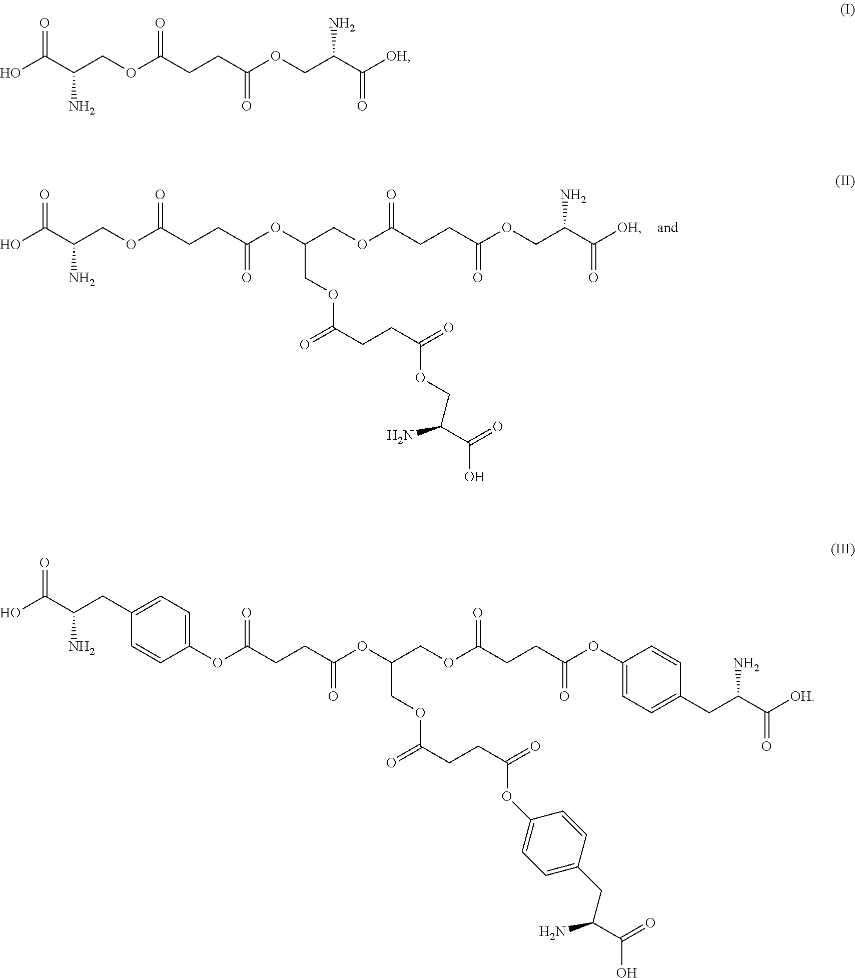 [SMILES: N[C@@H](COC(=O)CCC(=O)OC[C@H](N)C(=O)O)C(=O)O ; N[C@@H](COC(=O)CCC(=O)OCC(COC(=O)CCC(=O)OC[C@H](N)C(=O)O)OC(=O)CCC(=O)OC[C@H](N)C(=O)O)C(=O)O ; N[C@@H](Cc1ccc(OC(=O)CCC(=O)OCC(COC(=O)CCC(=O)Oc2ccc(C[C@H](N)C(=O)O)cc2)OC(=O)CCC(=O)Oc2ccc(C[C@H](N)C(=O)O)cc2)cc1)C(=O)O]

The TCA cycle intermediate or prodrug may include a structure represented by formula (IV):

A-β-hydroxybutyrate-B-β-hydroxybutyrate-A (IV),

in which A is an amino acid and B is a TCA cycle intermediate. In preferred embodiments, A is serine, and B is succinate.

The TCA cycle intermediate or prodrug may include a structure represented by formula (V):

C-D-E (V),

in which C is a first TCA cycle intermediate, D is a second TCA cycle intermediate, and E is an amino acid. In preferred embodiments, C is malate, D is succinate, and E is serine.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)0₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR⁰²; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R• (or the ring formed by taking two independent occurrences of R• together with their intervening atoms), are independently halogen, —(CH₂)₀₋₂R•, -(haloR•), —(CH₂)₀₋₂OH, —(CH₂)₀₋₂OR•, —(CH₂)₀₋₂CH(OR•)₂; —O(haloR•), —CN, —N₃, —(CH₂)₀₋₂C(O)R•, —(CH₂)₀₋₂C(O)OH, —(CH₂)₀₋₂C(O)OR•, —(CH₂)₀₋₂SR•, —(CH₂)₀₋₂SH, —(CH₂)₀₋₂NH₂, —(CH₂)₀₋₂NHR•, —(CH₂)₀₋₂NR•₂, —NO₂, —SiR•₃, —OSiR•₃, —C(O)SR•, —(C₁₋₄ straight or branched alkylene)C(O)OR•, or —SSR• wherein each R• is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R• include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁_₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R•, -(haloR•), —OH, —OR•, —O(haloR•), —CN, —C(O)OH, —C(O)OR•, —NH₂, —NHR•, —NR•, or —NO₂, wherein each R• is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R•, —(R•), —OH, —OR•, —O(haloR•, ), —CN, —C(O)OH, —C(O)OR•, —NH₂, —NHR•, —NR•, or —NO₂, wherein each R• is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The amino acid may be any naturally-occurring or non-naturally-occurring amino acid. Naturally-occurring amino acids include the following twenty amino acids that are encoded by the genetic code and incorporated into polypeptides by the translational machinery: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. Some naturally-occurring amino acids, such as selenocysteine and pyrrolysine, are found in polypeptides but are incorporated by alternative mechanisms. Other naturally-occurring amino acids, such as ornithine, citrulline, β-alanine, carnitine, γ-aminobutyrate, L-thyroxine, hydroxyproline, selenomethionine, and 2-aminoisobutyrate are not found in polypeptides. Non-naturally-occurring amino acids include amino acids that are not found in proteins or produced by cellular metabolic machinery, such as those described in Young and Schultz, Beyond the Canonical 20 Amino Acids: Expanding the Genetic Lexicon, J. Biol. Chem. 285(15):11039-11044 (2010); U.S. Pat. Nos. 7,566,555; and 9,488,660, each of which is incorporated herein by reference.

The compounds may include two or more TCA intermediates or prodrugs thereof attached via one or more linkers or backbone moieties. The backbone moiety may be a C₂₋₂₀ hydrocarbon moiety substituted with two or more groups selected from one or more of hydroxyl, amino groups, and carboxyl groups. The backbone moiety may be a polyol, such as a C₂-C₂₀ polyol, e.g., glycerol, erythritol, or xylitol. Alternatively or additionally, the two or more TCA intermediates or prodrugs thereof may be attached to each other directly.

The compounds may be represented by formula (VI):

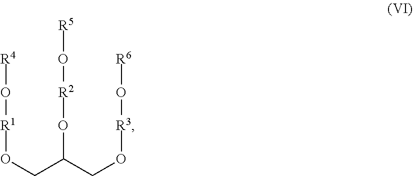

in which R¹, R², and R³ are TCA cycle intermediates or prodrugs thereof, and R⁴, R⁵, and R⁶ are amino acids. R¹, R², and R³ may be the same or different, and R⁴, R⁵, and R⁶ may be the same or different. R¹, R², and R³ may be succinate. R⁴, R⁵, and R⁶ may be serine, threonine, or tyrosine. If R⁴, R⁵, and R⁶ are serine, threonine, or tyrosine, they may be linked via the oxygen atom on their side chains, and the carboxyl group and amino group may be free and thus able to form COO⁻ and NH₃⁺ ions in aqueous solutions.

The compounds, including the capping moieties, may include one or more atoms that are enriched for an isotope. For example, the compounds may have one or more hydrogen atoms replaced with deuterium or tritium. Isotopic substitution or enrichment may occur at carbon, sulfur, or phosphorus atoms as well. The compounds may be isotopically substituted or enriched for a given atom at one or more positions within the compound, or the compounds may be isotopically substituted or enriched at all instances of a given atom within the compound.

The compounds may have an octanol:water partition coefficient of less than 0.1, less than 0.01, less than 0.001, less than 0.0001, less than 0.0001, less than 0.00001, or less than 0.000001.

The solubility of TCA cycle intermediates can be increased by covalently linking capping moieties to such molecules. In particular, it is advantageous to add capping moieties as substituents on the hydroxyl groups of TCA cycle intermediates. Such capped-alcohol molecules have improved solubility and do not have offensive odors.

Thus, methods of the invention may provide compounds that include a TCA cycle intermediate or prodrug thereof and covalently linked to two or more capping moieties. For example, the compounds may include a TCA cycle intermediate linked to two, three, four, five, or six capping moieties.

The TCA cycle intermediate or prodrug thereof may be citrate, cis-aconitate, D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate. The TCA cycle intermediate may have L or R chirality. Compositions including such compounds may include only L-forms, only R-forms, or racemic mixtures of L- and R-forms of the TCA cycle intermediate.

The two or more capping moieties may be the same, or they may be different. The capping moieties may be polyols, such as C₂-C₂₀ polyols, amino acids, or other TCA cycle intermediates or prodrugs thereof. The compound may have two capping moieties, both of which are glycerol. The compound may have two capping moieties, with one being malate and the other being serine.

The capping moieties may be linked by any atoms on the TCA cycle intermediate or prodrug thereof. Preferably, capping moieties are substituted onto hydroxyl groups and attached via alkoxy linkages. Preferably, a capping moiety is substituted onto the hydroxyl group of each of the terminal carbon atoms in the carbon skeleton of the TCA cycle intermediate or prodrug thereof. The TCA cycle intermediate or prodrug thereof may be represented by one of formulas (VII), (VIII), (IX), (X), (XI), and (XII):

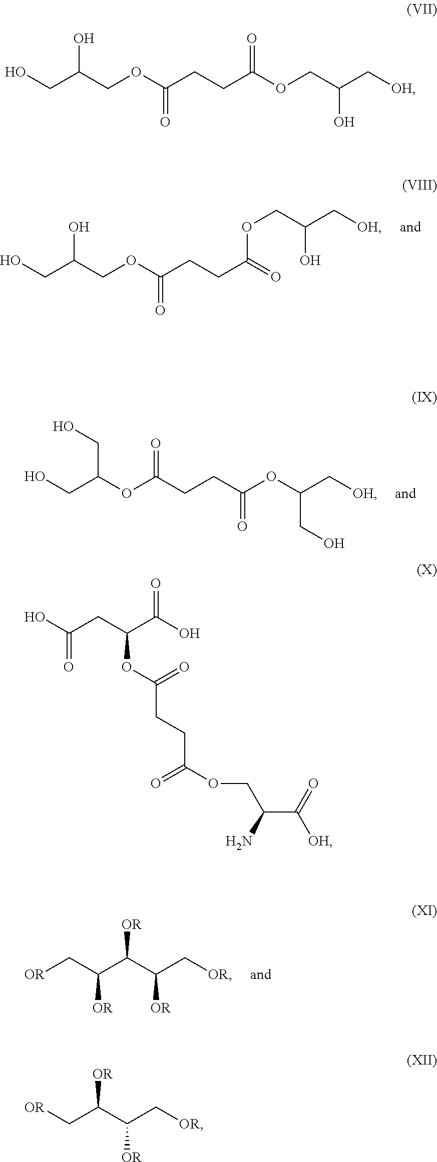

in which R is

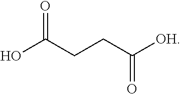

In certain embodiments, the compounds include a polyol, a TCA cycle intermediate or prodrug thereof covalently linked to the polyol, and an amino acid covalently linked to the TCA cycle intermediate or prodrug thereof. Each of the polyol, the CA cycle intermediate or prodrug thereof, and the amino acid may be as described above in reference to such components. Preferably, the polyol is glycerol, the TCA intermediates or prodrugs thereof is succinate, and the amino acid is serine. The polyol may be linked via a terminal hydroxy group or an internal hydroxy group. For example, glycerol may linked to the TCA cycle intermediate or prodrug thereof via a hydroxy group on its first, second, or third carbon. The compound may be represented by one of formulas (XIII) and (XIV):

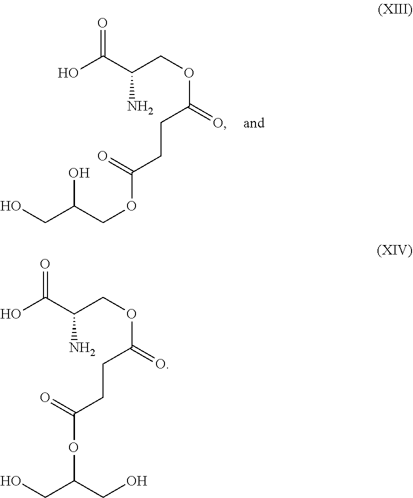

In certain embodiments, the TCA cycle intermediate is α-ketoglutarate. Optionally, the amino acid is serine. Optionally, the polyol is glycerol. In certain embodiments, the compound is represented formula (XV):

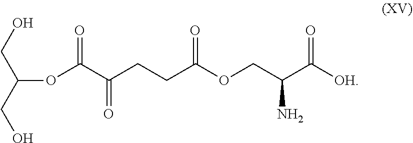 [SMILES: N[C@@H](COC(=O)CCC(=O)C(=O)OC(CO)CO)C(=O)O]

In other embodiments, the TCA cycle intermediate is β-hydroxybutyrate. Optionally, the amino acid is serine. Optionally, the polyol is glycerol. In certain embodiments, the compound is represented formula (XVI):

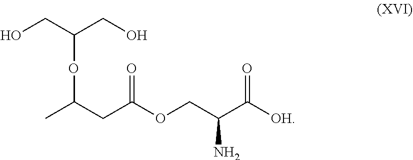 [SMILES: CC(CC(=O)OC[C@H](N)C(=O)O)OC(CO)CO]

In other embodiments, methods of the invention provide compounds including citrate or citric acid, prodrugs, analogs, derivatives, or salts thereof, and one or more amino acids. In certain embodiments, the compound includes a plurality of amino acids, e.g., at least two or three amino acids. In preferred embodiments, the compound includes three amino acids. Numerous different types of amino acids can be conjugated to the citrate. The amino acids may be any naturally-occurring or non-naturally-occurring amino acids or combinations thereof (e.g., all naturally occurring, all non-naturally occurring, or a combination of naturally and non-naturally occurring amino acids). The amino acids may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. The amino acid may be serine and tyrosine. In certain embodiments, the amino acid is serine and the compound includes three serines. An exemplary compound is represented formula (XVII):

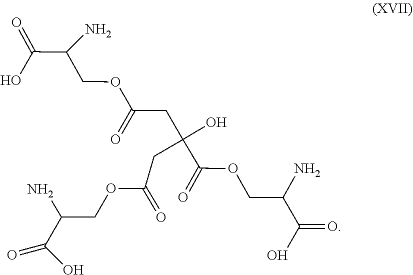 [SMILES: NC(COC(=O)CC(O)(CC(=O)OCC(N)C(=O)O)C(=O)OCC(N)C(=O)O)C(=O)O]

In certain embodiments, the TCA cycle intermediate or prodrug thereof is selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate. In particular embodiments, the TCA cycle intermediate or prodrug thereof is citrate. In certain embodiments, the polyol is glycerol. In certain of such embodiments, the composition comprises a plurality of citrate molecules covalently bound to one or more glycerol molecules. In a preferred embodiment, the composition comprises a plurality of citrate molecules, at least one of which is covalently bound to a plurality of glycerol molecules. A preferred compound is a compound of Formula (XVIII):

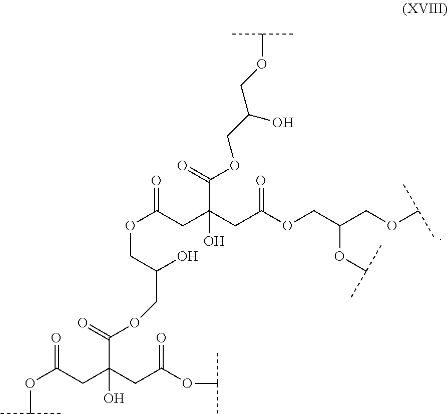

The methods of invention may provide compositions comprising a TCA cycle intermediate anhydride or polymer or pharmaceutically acceptable salt or prodrug thereof in a therapeutically effective amount to treat a condition associated with altered TCA cycle metabolism in a subject. The TCA cycle intermediate or pharmaceutically acceptable salt or polymer or prodrug thereof may be selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate.

In certain embodiments, the prodrug comprises one or more polyols. In other embodiments, the prodrug comprises one or more amino acids. In certain embodiments, the prodrug comprises one or more polyols and one or more amino acids. In certain embodiments, the composition is a polymer of a TCA cycle intermediate, e.g., one or more repeating units of a TCA cycle intermediate monomer.

In an exemplary embodiment, the TCA cycle intermediate anhydride or polymer or pharmaceutically acceptable salt or prodrug thereof is citric acid anhydride or polymer or a pharmaceutically acceptable salt or prodrug thereof. In certain embodiments, the citric acid anhydride is selected from the group consisting of a symmetrical citric acid anhydride, an asymmetrical citric acid anhydride, an intermolecular citric acid anhydride, and a combination thereof. Examples of each are shown below as formulas (XIX), (XX), and (XXI):

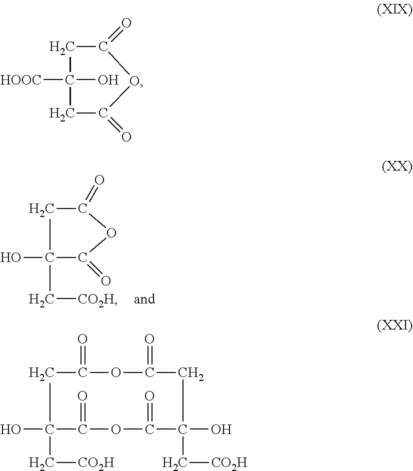

In certain embodiments, the citric acid anhydride is a prodrug of citric acid anhydride. Such exemplary prodrugs may comprise one or more polyols. In other embodiments, the citric acid anhydride prodrug comprises one or more amino acids. In certain embodiments, the citric acid anhydride prodrug comprises one or more polyols and one or more amino acids. In certain embodiments, the composition is a citric acid anhydride polymer, e.g., one or more repeating units of a citric acid anhydride monomer.

In certain embodiments, the composition is formulated for oral or gastric administration. In certain embodiments, the composition is formulated as a single unit dose.

In methods of the invention, one or more compounds described above may be provided as a pharmaceutical composition. A pharmaceutical composition containing the compounds may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. 2003/0232877, incorporated by reference herein in their entirety.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Compounds may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

Compounds may be provided as intraocular formulations. Intraocular formulations include any formulation suitable for delivery of an agent to the eye. For example and without limitation, intraocular formulations include aqueous gels, contact lenses, dendrimers, emulsions, emulsions, eye drops, implants, in situ thermosensitive gels, liposomes, microneedles, nanomicelles, nanoparticles, nanosuspensions, ointments, and suspensions. Ocular formulations are known in the art and describe in, for example, Patel, A., et al., Ocular drug delivery systems: An overview, World J Pharmacol. 2013; 2(2): 47-64. doi:10.5497/wjp.v2.i2.47; U.S. Pat. No. 9,636,347; U.S. Publication Nos. 2017/0044274 and 2009/0148527; and International Publication No. WO 2015/105458, the contents of each of which are incorporated herein by reference.

The methods of the invention include providing a composition of the invention, as described above, to a subject having or at risk of developing LHON. Providing may include administering the composition to the subject. The composition may be administered by any suitable means, such as intraocularly, orally, intravenously, enterally, parenterally, dermally, buccally, topically (including transdermally), by injection, intravenously, nasally, pulmonarily, and with or on an implantable medical device (e.g., stent or drug-eluting stent or balloon equivalents).

The composition may be provided under any suitable dosing regimen. For example, the composition may be provided as a single dose or in multiple doses. Multiple doses may be provided in provided separated by intervals, such as 4 hours, 6 hours, 8 hours, 12 hours, 24 hours, 2 days, 3 days, 4 days, 5 days, 1 week, 2 weeks, 3 weeks, 4 weeks, or more. Multiple doses may be provided within a period of time. For example, multiple doses may be provided over a period of 1 day, 2 days, 3 days, 4 days, 5 days, 1 week, 2 weeks, 3 weeks, 4 weeks, or more.

The methods may include providing conjugated TCA cycle intermediate, such as any of those described above, in combination with a second therapy. For example and without limitation, the second therapy may include providing one or more of brimonidine, curcumin, glutathione, elamipretide, idebenone, and minocycline; near infrared light treatment; gene therapy, for example, using a viral vector; or avoidance of an optic nerve toxin, such as alcohol, tobacco, or vitamin B₁₂.

The subject may be a human subject that has or is at risk of developing LHON. The subject may a mitochondrial mutation associated with LHON. For example, the subject may have a mutation in MT-ND1, MT-ND4, MT-ND4L, and MT-ND6. The subject may have begun to experience vision loss. For example, the subject may have vision of 20/40, 20/80, 20/100, 20/150, 20/20, or worse. The subject may have begun to experience vision loss within a period of time prior to initiation of treatment. For example and without limitation, the methods may include providing a compound described above within 1 week, 2 weeks, 3 weeks, 4 weeks, 6 weeks, 8 weeks, 10 weeks, or 12 weeks of commencement of vision loss. The subject may have begun to experience vision loss in one eye or in both eyes.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of treating or preventing Leber's hereditary optic neuropathy in a subject, the method comprising providing to a subject having or at risk of developing Leber's hereditary optic neuropathy a composition comprising a compound, wherein the compound comprises:
one or more TCA cycle intermediates or prodrugs thereof; and
one or more capping moieties.

2. The method of claim 1, wherein the one or more capping moieties are selected from the group consisting of an amino acid and a polyol.

3. The method of claim 2, wherein the amino acid is naturally-occurring.

4. The method of claim 3, wherein the amino acid is serine or tyrosine.

5. The method of claim 2, wherein the amino acid is not naturally-occurring.

6. The method of claim 2, wherein the polyol is glycerol.

7. The method of claim 2, wherein the one or more TCA cycle intermediates or prodrugs thereof are selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate.

8. The method of claim 7, wherein one or more TCA cycle intermediates or prodrugs thereof comprise succinate.

9. The method of claim 8, wherein the compound is represented by a formula selected from the group consisting of (I), (II), (III), (IV), (V), (VI), (VII), (VIII), (IX), (X), (XIII), and (XIV): 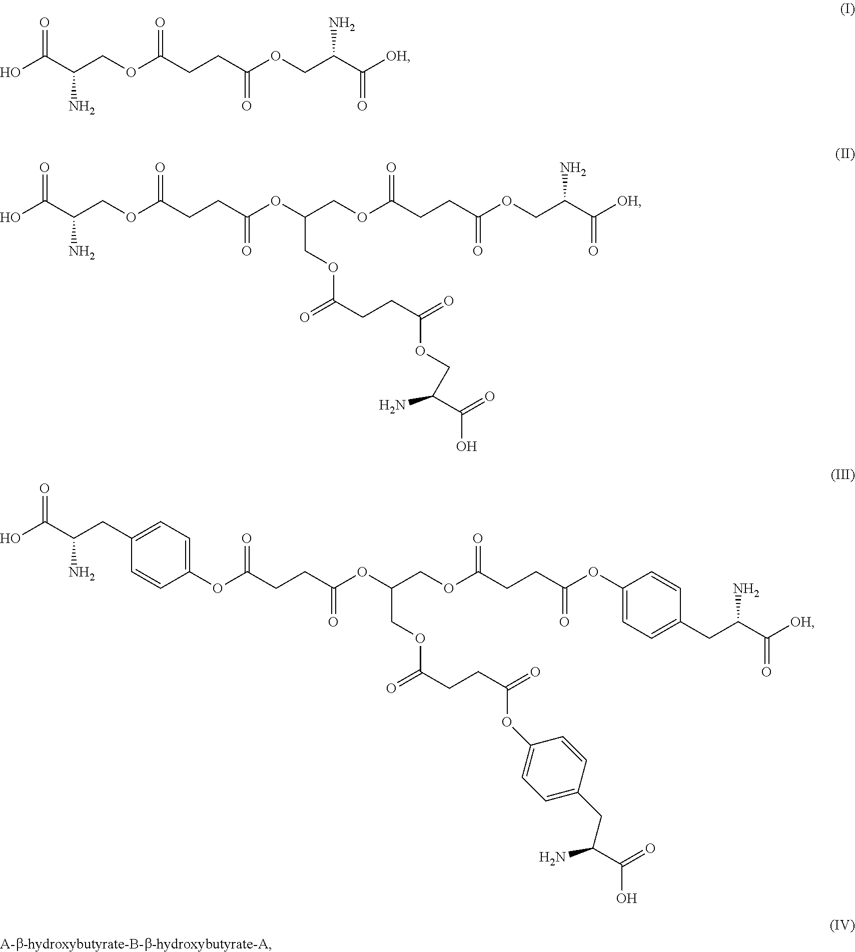
wherein:
A is serine; and
B is succinate,
C-D-E (V),
wherein:
C is malate;
D is succinate; and
E is serine, 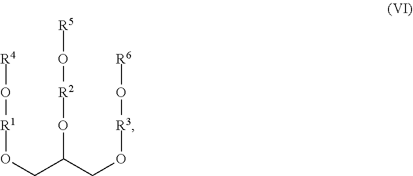
wherein:
R¹, R², and R³ are succinate;
R⁴, R⁵, and R⁶ are serine; and
R⁴, R⁵, and R⁶ are linked via oxygen atoms on their side chains to R¹, R², and R³, respectively,

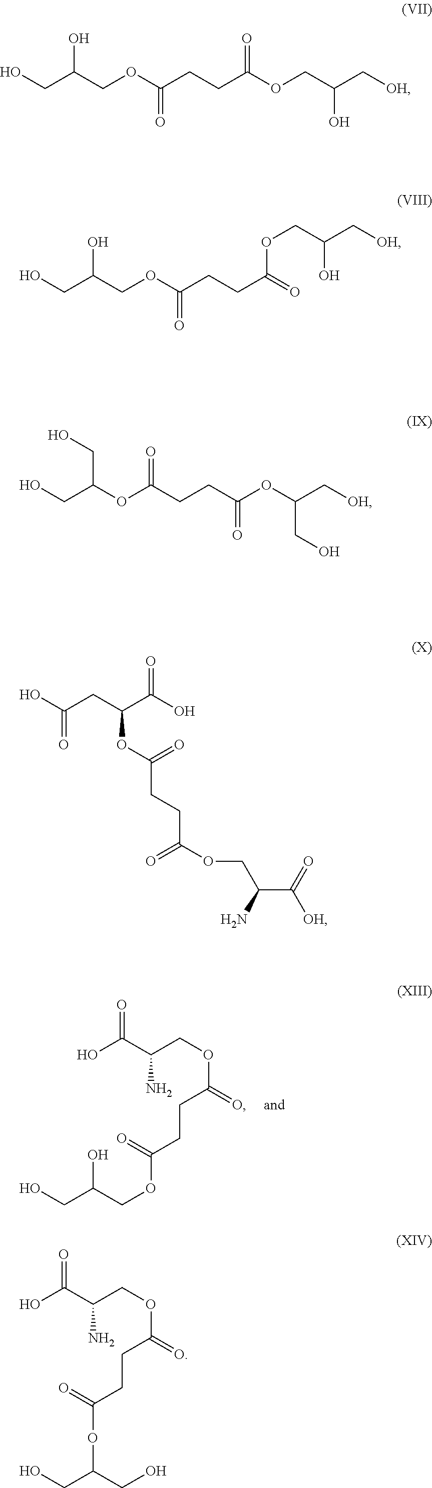 [SMILES: O=C(CCC(=O)OCC(O)CO)OCC(O)CO ; O=C(CCC(=O)OCC(O)CO)OCC(O)CO ; O=C(CCC(=O)OC(CO)CO)OC(CO)CO ; N[C@@H](COC(=O)CCC(=O)O[C@@H](CC(=O)O)C(=O)O)C(=O)O ; N[C@@H](COC(=O)CCC(=O)OCC(O)CO)C(=O)O ; N[C@@H](COC(=O)CCC(=O)OC(CO)CO)C(=O)O]

10. The method of claim 7, wherein one or more TCA cycle intermediates or prodrugs thereof comprise citrate.

11. The method of claim 10, wherein the compound is represented by a formula selected from the group consisting of (XVII) and (XVIII):

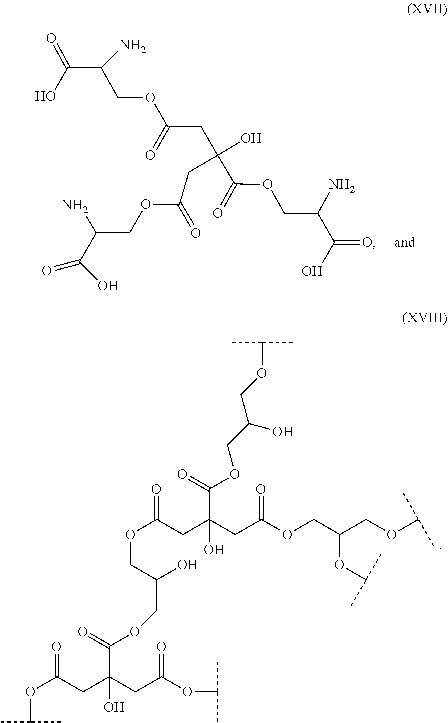

12. The method of claim 7, wherein one or more TCA cycle intermediates or prodrugs thereof comprise α-ketoglutarate

13. The method of claim 12, wherein the compound is represented by formula (XV):

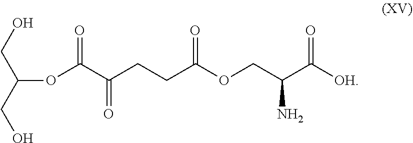 [SMILES: N[C@@H](COC(=O)CCC(=O)C(=O)OC(CO)CO)C(=O)O]

14. The method of claim 7, wherein one or more TCA cycle intermediates or prodrugs thereof comprise β-hydroxybutyrate.

15. The method of claim 14, wherein the compound is represented by formula (XVI):

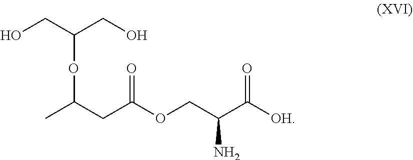 [SMILES: CC(CC(=O)OC[C@H](N)C(=O)O)OC(CO)CO]

16. The method of claim 7, wherein one or more TCA cycle intermediates comprise an anhydride or polymer.

17. The method of claim 2, wherein the compound comprises two or more TCA cycle intermediates or prodrugs thereof.

18. The method of claim 2, wherein the compound comprises two or more capping moieties.

19. The method of claim 1, wherein the compound is provided intraocularly.

20. The method of claim 1, wherein the compound is provided orally or intravenously.
